(19) United States
(12) Patent Application Publication
(10) Pub. No.: US 2012/0178672 A1
(43) Pub. Date: Jul. 12, 2012

(54) Compositions and Methods for Sparing Muscle in Renal Insufficiency and During Hemodialysis

(76) Inventors: Robert R. Wolfe, Little Rock, AR (US); John P. Troup, Plymouth, MN (US)

(21) Appl. No.: 12/727,051

(22) Filed: Mar. 18, 2010

Related U.S. Application Data
(60) Provisional application No. 61/161,296, filed on Mar. 18, 2009.

(51) Int. Cl. A61K 38/02; A61P 3/02; A61K 31/4172; A61P 21/00; A61K 31/70; A61K 31/202

(52) U.S. Cl. 514/4.8; 514/1.1; 514/23; 514/560; 514/400

(57) ABSTRACT

A nutritional composition and method of use that improves the net balance in skeletal muscle by targeting both the synthetic and breakdown processes. The disclosed composition provides for improved protein intake to increase skeletal muscle protein accretion in stressed patients who are at risk for the development of renal insufficiency by stimulating protein synthesis.

### CROSS REFERENCE TO RELATED APPLICATION(S)

This application claims the benefit of U.S. Provisional Patent Application Ser. No. 61/161,296 filed Mar. 18, 2009 the entire disclosure of which is herein incorporated by reference.

### BACKGROUND

1. Field of the Invention

This disclosure relates to the field of nutritional compositions. In particular, to the field of nutritional compositions including amino acids and specifically those for stressed patients who are at risk for developing renal insufficiency, who have renal insufficiency, or are being treated by hemodialysis.

2. Description of the Related Art

Protein synthesis is the process of building or making (i.e., “synthesizing”) new body proteins. For skeletal muscle, protein synthesis entails the synthesis of mitochondrial, sarcoplasmic and myofibrillar proteins, i.e., the protein components that comprise skeletal muscle. Of these protein components, myofibrillar proteins are those most responsible for the functional component of skeletal muscle. Conversely, protein breakdown is the process by which proteins are degraded by the body. This is accomplished by several distinct processes, or pathways, and the exact process by which myofibrillar protein is degraded is an ongoing area of research and scientific advancement.

The relationship between protein synthesis and protein breakdown in the body at a given time is referred to as the “net balance” (net balance=protein synthesis−protein breakdown). A positive net balance (or net protein synthesis) refers to muscle in an anabolic, or building, state. In contrast, a negative net balance refers to muscle in a catabolic state, i.e., experiencing overall protein degradation.

Through a cycle of protein building and protein degradation, muscle plays a key role in whole-body protein metabolism by serving as the principal “back-up” reservoir for the amino acids required to maintain protein synthesis in vital tissues and organs. In the absence of sufficient amino acids levels derived from food intake, skeletal muscle serves as the body's precursor reservoir for both vital proteins and hepatic gluconeogenic precursors. This ability of the body to obtain amino acids from skeletal muscle becomes important when the body enters a stressed state, such as sepsis, advanced cancer, congestive heart failure, chronic kidney disease (“CKD”), or any severe injury, such as burns. During these “stressed states” the loss of muscle mass is an important predictor of mortality and morbidity. In all of these stressed states, renal insufficiency is common, and often leads to renal failure, i.e., a situation in which the kidneys fail to function adequately. Renal failure is a common occurrence in these “stressed states” as the result of a sudden interruption in the blood supply to the kidney or as a result of toxic overload in the kidneys.

Generally, renal failure is measured in five stages, which are calculated using a patient's GFR, or glomerular filtration rate (GFR). Stage 1 disease is mildly diminished renal function, with few overt symptoms, defined by a normal GFR (greater than 90 ml/min per 1.73 m² of body surface area) and persistent albuminuria. Stage 2 and 3 disease need increasing levels of supportive care from their medical providers to slow and treat their renal dysfunction. These stages are defined by a GFR between 60 to 89 ml/min per 1.73 m2 and persistent albuminuria (2.8%), and a GFR between 30 and 59 ml/min per 1.73 m2 (3.7%), respectively. Stage 4 disease is defined by a GFR between 15 and 29 ml/min per 1.73 m2 (0.13%), and Stage 5 disease is a GFR of less than 15 ml/min per 1.73 m2 or end-stage renal disease (0.2%). Patients in these stages usually require active treatment in order to survive. Stage 5, in particular, is considered a severe illness and requires some form of renal replacement therapy (i.e., dialysis) or kidney transplant whenever feasible.

One method of treating renal failure is hemodialysis, a method that removes waste products such as potassium and urea via a filtration mechanism from the blood after renal failure. Once renal failure requiring dialysis has occurred, the loss of lean body mass is a strong predictor of death and is inversely correlated with the outcome of death.

One reason attributable to the loss of lean body mass during dialysis is the high prevalence (approximately 33%) of protein-energy malnutrition in renal insufficient patients receiving maintenance hemodialysis (“HD”). While skeletal muscle degradation, or a negative net balance, can be found in each stage of renal failure due to alterations in protein metabolism, HD only exacerbates the patient's catabolic state. Common causes for this malnutrition in dialyzed patients are decreased energy or protein intake, the catabolic stimulus of HD (i.e., the ability of HD to stimulate the breakdown of proteins into amino acids and simple derivative compounds), and the loss of nutrients, particularly amino acids, during HD.

The deleterious effects of renal insufficiency have become evident to those of skill in the art in the study of CKD patients. Alterations in protein metabolism are responsible for the loss of skeletal mass in CKD patients. In the fasted state, CKD patients exhibit a significantly lower rate of muscle protein synthesis than their age matched healthy counterparts as early as stage 3. However, the greatest alteration in protein metabolism for these patients results from the continued insult of HD, which increases protein turnover and results in an increased net protein catabolism. The loss of nitrogen from skeletal muscle supports an increase in hepatic (liver) protein synthesis, most notably that of albumin and fibrinogen. Further, amino acids derived from skeletal muscle are utilized for the intra-dialytic synthesis of acute phase proteins. Thus, the increased central demand for amino acids resulting from HD is a primary stimulus for increased degradation of skeletal muscle.

In addition to skeletal muscle degradation, a second catabolic influence on skeletal muscle in renal insufficiency is ascribed to cytokine activation during HD. The term “cytokine” refers to small secreted proteins which mediate and regulate immunity and inflammation. The largest group of cytokines stimulates immune cell proliferation and differentiation. Cytokines which are produced predominantly by activated immune cells and which are involved in the amplification of inflammatory reactions are called pro-inflammatory cytokines. These pro-inflammatory cytokines include Interleukin-1 (IL-1), IL-6, and tumor necrosis factor (TNF-α). Briefly and generally, IL-1 activates T cells, IL-6 stimulates proliferation and differentiation of β cells, and TNF-α is involved in systemic inflammation the stimulation of acute phase protein synthesis. While cytokines are important to the immune and inflammatory process, they are chronically elevated in renal-insufficient patients, indicating a sustained inflammatory state. The additional complication of chronically elevated cytokines is their link to increased muscle protein breakdown.

Renal insufficiency is a state of microinflammation, which is further exacerbated by cytokine production during HD. Both in vitro and in vivo data suggest that cytokine production during HD is caused by (a) direct contact of peripheral blood mononuclear cells (PBMC) with the dialysis membrane, (b) active complement fragments generated during HD, and (c) transport of bacterial derived material from the dialystate into the blood compartment. Human skeletal muscle cells have the inherent ability to express a variety of cytokines, including IL-6, which has been shown to activate proteolytic pathways in muscle. Cytokines in general stimulate proteolysis by increasing ubiquitin conjugation (a primary pathway for the degradation of muscle proteins). It has been demonstrated that IL-6 is released from skeletal muscle during HD. Further, evidence has been found to support the role of IL-6 in the activation of genes promoting catabolism and the subsequent loss of amino acids from the muscle, as well as increased synthesis of albumin and fibrogen during HD. Thus, cytokines are released from both PBMC and muscle during HD and contribute to the muscle catabolism of HD. By extension, the ability to ameliorate hypercytokinemia during HD in renal insufficient patients would serve to improve protein balance in skeletal muscle

It is known in the art that severely stressed and critically ill patients benefit from high protein intake to counteract, in part, the rapid loss of muscle mass. Conversely, low protein diets have long been recommended for renal insufficient patients, since increased protein intake adversely affects blood acidity and urea production. Therefore, it has been difficult in the art to develop a technique to provide the necessary protein nutrition to patients with renal failure, as attempts to optimize protein intake are hampered by the inherent need to minimize potentially harmful by-products. Thus, there is a need in the art for a way to meet protein requirements for muscle metabolism using a format which provides a greater response of muscle protein anabolism than the intake of high quality protein alone, while not adversely affecting blood acidity and urea production. Further, a nutritional intervention which minimizes the deleterious effects of cytokine production would also be greatly beneficial and fill in a hole in the art, as such nutritional intervention would ameliorate the catabolic drive of muscle protein breakdown. Thus, there exists in the art the need for a nutritional composition that has the ability to optimize nitrogen intake to maximize the response of skeletal muscle, while minimizing those problems inherent to patients who develop renal insufficiency.

### SUMMARY

Due to these and other problems in the art, disclosed herein are nutritional compositions and methods of use for treating patients that improves the net balance in skeletal muscle by targeting both the synthetic and breakdown processes. The disclosed compositions generally provide for improved protein intake to increase skeletal muscle protein accretion in stressed patients who are at risk for the development of renal insufficiency by stimulating protein synthesis. The disclosed compositions can also provide for a nutritional formula designed to ameliorate the loss of protein in patients during HD. Further, the disclosed composition can be used to slow, reduce, or prevent the loss of skeletal muscle mass prevalent in such patients. In addition, the disclosed compositions may prevent or reduce the increase in blood acidity and urea, while reducing the deleterious effects of increased cytokine production.

There is described herein, among other things, a composition of matter comprising: an EAA blend, said EAA blend including: valine, threonine, isoleucine, leucine, lysine, phenylalanine, and methionine, and not including glutamine or alanine; eicosapentaenoic acid (EPA); a macronutrient blend, said macronutrient blend comprising macronutrients selected from the group consisting of protein, carbohydrate, and fat; and a buffering agent.

In an embodiment, the EAA blend further comprises arginine and histidine.

In an embodiment of the composition: histidine comprises 1-6% of said EAA blend; isoleucine comprises 6-15% of said EAA blend; leucine comprises 15-40% of said EAA blend; lysine comprises 10-25% of said EAA blend; methionine comprises 1-5% of said EAA blend; phenylalanine comprises 5-15% of said EAA blend; threonine comprises 5-15% of said EAA blend; valine comprises 5-20% of said EAA blend; and arginine comprises 5-15% of said EAA blend.

In an embodiment the composition further comprises citrulline.

In an embodiment of the composition, carbohydrate comprises 30-60% of said macronutrient blend; fat comprises 10-25% of said macronutrient blend; and protein comprises 25-75% of said macronutrient blend.

In an embodiment of the composition said buffering agent comprises sodium bicarbonate.

In an embodiment The composition is comprised of: about 15 grams of said EAA blend; about 1 to about 15 grams of said macronutrient blend; about 250 to about 1500 milligrams of EPA; and about 100 to about 700 milligrams of buffering agent.

In another embodiment said macronutrient blend comprises protein peptides and/or whey protein.

In another embodiment, the composition is part of a food product such as, but not limited to, a drink.

In another embodiment, the composition is part of a pharmaceutical preparation such as, but not limited to, a tablet, liquid suspension, nasal spray, or suppository.

There is also described herein, a composition of matter consisting essentially of: valine; threonine; isoleucine; leucine; lysine; phenylalanine; methionine; histidine; and arginine.

In an embodiment histidine comprises 1-6% of said composition; isoleucine comprises 6-15% of said composition; leucine comprises 15-40% of said composition; lysine comprises 10-25% of said composition; methionine comprises 1-5% of said composition; phenylalanine comprises 5-15% of said composition; threonine comprises 5-15% of said composition; valine comprises 5-20% of said composition; and arginine comprises 5-15% of said composition.

There is also described herein a composition of matter consisting essentially of: an EAA blend consisting of: valine, threonine, isoleucine, leucine, lysine, phenylalanine, and methionine; histidine; and arginine; eicosapentaenoic acid (EPA); a macronutrient blend consisting of protein; carbohydrate; and fat; and a buffering agent.

In an embodiment of the composition, histidine comprises 1-6% of said EAA blend; isoleucine comprises 6-15% of said EAA blend; leucine comprises 15-40% of said EAA blend; lysine comprises 10-25% of said EAA blend; methionine comprises 1-5% of said EAA blend; phenylalanine comprises 5-15% of said EAA blend; threonine comprises 5-15% of said EAA blend; valine comprises 5-20% of said EAA blend; and arginine comprises 5-15% of said EAA blend.

In an embodiment of the composition, carbohydrate comprises 30-60% of said macronutrient blend; fat comprises 10-25% of said macronutrient blend; and protein comprises 25-75% of said macronutrient blend. The buffering agent may also comprise sodium bicarbonate.

There is also described herein a method of inhibiting skeletal muscle degradation during renal failure, the method comprising: administering to said individual a therapeutically effective amount of a composition comprising: an EAA blend comprising: valine, threonine, isoleucine, leucine, lysine, phenylalanine, and methionine; histidine; and arginine; eicosapentaenoic acid (EPA); a macronutrient blend consisting of protein; carbohydrate; and fat; and a buffering agent; and administering hemodialysis to said individual.

There is also described herein a composition of matter consisting essentially of: an EAA blend consisting of: valine, threonine, isoleucine, leucine, lysine, phenylalanine, and methionine; histidine; and arginine; and eicosapentaenoic acid (EPA).

### BRIEF DESCRIPTION OF THE DRAWINGS

FIG. 1 provides a graphical depiction of the net phenylalanine uptake (reflecting protein synthesis) 3.5 hours after 15 g EAA or 15 g of whey in elderly test subjects.

FIG. 2A provides a graphical depiction of urea production after ingestion of 12 g EAAs.

FIG. 2B provides a graphical depiction of alanine concentration after ingestion of 12 g EAAs.

FIG. 3 provides a graphical depiction of increases in lean body mass in 12 elderly impaired glucose tolerant subjects after 12 weeks of EAA administration.

FIG. 4A provides a graphical depiction of gain in muscle strength, as determined by 1 Repetition Maximum in elderly insulin resistant subjects after 12 weeks of EAA supplementation.

FIG. 4B provides a graphical depiction of gain in muscle strength, as determined by function of walking speed in elderly insulin resistant subjects after 12 weeks of EAA supplementation.

FIG. 5 provides a graphical depiction of protein kinetics before and during HD.

### DESCRIPTION OF PREFERRED EMBODIMENT(S)

The disclosed compositions are based, in part, on a ratio of essential and conditionally essential amino acids. When taken in adequate amounts and a prescribed ratio, this formulation generally leads to a stimulation of net protein synthesis, the accretion of muscle mass and improved muscle function. In addition, the composition may further include components which address key problems associated with renal patients requiring HD, namely the potential for increased blood acidity and inflammatory responses.

In order to lay a proper foundational understanding for the disclosed nutritional composition and method of use to be understood, the following terms are defined.

The term “essential amino acids,” as it is used herein, refers to 8 amino acids that can not be produced in the body, but are required for the manufacture of proteins in the body. In essence, they are essential to the body, but must be derived from dietary intake. The 8 essential amino acids are: tryptophan, valine, threonine, isoleucine, leucine, lysine, phenylalanine, and methionine. Histidine is an amino acid that is considered conditionally essential, in that it is often limited by dietary intake and required in greater quantities by an individual in a stressed state. Similarly, arginine is conditionally essential and under certain conditions is not produced in a sufficient state. In an embodiment, the nutritional composition disclosed herein utilizes 7 of the 8 essential amino acids, which may be provided alone or in addition to histidine and/or arginine as an amino acid blend which is generally referred to herein as the “EAA blend” or “EAAs.” In some embodiments, the EAA blend may also include other conditionally essential amino acids such as, but not limited to tyrosine or cysteine. However, this is generally not preferred.

The term eicosapentaenoic acid (“EPA”), as it is used herein, refers to a long-chain polyunsaturated omega-3, or n-3, fatty acid. This fatty acid is a major component of fish oil. EPA is utilized in an embodiment of the composition, in part, for its role in the reduction of cytokine production and muscle protein breakdown.

In one embodiment, the composition is comprised of an amino acid blend formed of EAAs and conditionally essential amino acids (which are often jointly referred to as EAAs), a range of macronutrients, an eicosapentaenoic acid (“EPA”), and a buffering agent.

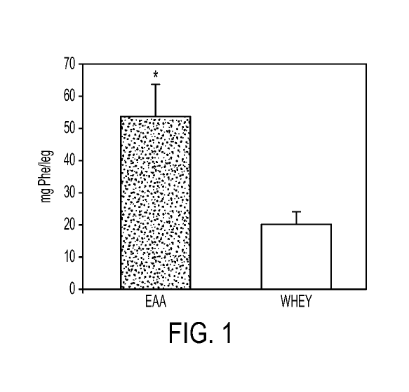

The EAAs in an embodiment of the composition are essential amino acids derived from free-form amino acids, protein peptides or high quality protein sources known to those of skill in the art. Generally, the EAA aspect of the disclosed composition has several advantages over traditional and existing formulas. First, as depicted in FIG. 1, the EAA aspect of the disclosed composition generally results in a greater stimulation, per gram intake, of muscle protein anabolism over intact quality proteins that are commonly ingested by most patients. Second, by providing only EAA, the disclosed composition makes use of existing non-essential amino acids present in the body. These non-essential amino acids, particularly alanine and glutamine, are the primary precursors of urea.

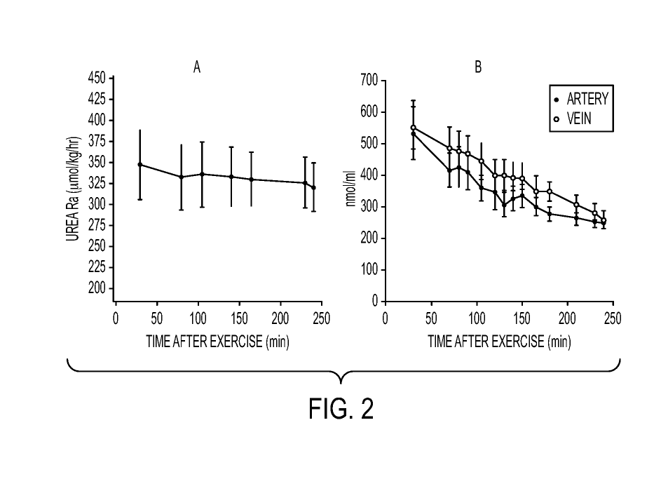

Compositions such as those discussed herein minimize the availability of non-essential amino acids by stimulating their incorporation into protein, thus minimizing their conversion to urea. This aspect of the disclosed composition is depicted in the chart of FIG. 2. Thus, in an embodiment, the composition itself and specifically the EAA blend therein, does not include non-essential amino acids and specifically does not include alanine or glutamine. As indicated, depending on embodiment, the EAA blend may include or not include conditionally essential amino acids such as, but not limited to, arginine and histidine. However, throughout this disclosure, references to the EAA blend will generally indicate a blend that includes valine, threonine, isoleucine, leucine, lysine, phenylalanine, methionine, histidine, and arginine.

In one embodiment of the nutritional composition, it is contemplated that the EAA blend in the composition is comprised of 1-6% of the amino acid histidine, 6-15% of the amino acid isoleucine, 15-40% of the amino acid leucine, 10-25% of the amino acid lysine, 1-5% of the amino acid methionine, 5-15% of the amino acid phenylalanine, 5-15% of the amino acid threonine, 5-20% of the amino acid valine, and 5-15% of the amino acid arginine and/or its precursor citrulline based on total protein content. While these percentages of EAA are disclosed, any percentage or combination of EAAs known to those of skill in the art that increases skeletal muscle protein synthesis is contemplated in this disclosure. In an embodiment it is contemplated that about 15 g of the EAA mixture would be provided per dose although larger or smaller doses may be provided based on the specific individual and this can include doses of about 30 g or larger or about 10 g or smaller.

The macronutrients of the disclosed composition include proteins, carbohydrates and fat components known to those of skill in the art for inclusion in nutritional compositions. In one embodiment, the contemplated range of macronutrients is 30-60% carbohydrate, 10-25% fat, and 25-75% protein; however any range of percentages known to those of skill in the art of these macronutrients for inclusion in nutritional supplements is contemplated in this disclosure. In one alternative embodiment, the protein component of the macronutrient element of the disclosed composition includes 1-15 grams of protein peptides. In another alternative embodiment, the protein component of the macronutrient element of the disclosed composition includes 1-15 grams of whey protein.

The EPA of an embodiment of the composition is an omega-3 fatty acid. Generally, inclusion of the EPA in the composition provides benefits in two ways. First, EPA is generally expected to counteract the chronic inflammatory state by reducing plasma cytokine concentrations and reactive oxygen species. As noted previously in this disclosure, forms of serious illness in which patients are at risk for the development of renal failure generally have some degree of an inflammatory response which contributes to the catabolic response of muscle. Accordingly, it has been demonstrated in the art that HD leads to substantial inflammatory response in skeletal muscle and circulation. Thus, the EPA component of the disclosed composition is expected to reduce the inflammatory response and reduce cytokine influence on protein breakdown.

Second, EPA generally has a direct inhibitory effect on muscle protein breakdown. Thus, a synergistic effect is generally expected between the EPA component and the amino acid component, since the anabolic aspect of EAAs is largely a stimulation of muscle protein synthesis. As the net balance, and ultimately the accumulation of muscle protein, is the result of a positive difference between protein synthesis and protein breakdown, an increase in muscle protein synthesis combined with a decrease in muscle protein breakdown increases the likelihood of a positive net protein balance in skeletal muscle. In other words, the inclusion of EAAs stimulates muscle protein synthesis while the inclusion of EPA slows the breakdown of muscle protein. The net protein balance is improved by enhancing each metabolic process. In one embodiment of the disclosed composition, it is contemplated that the composition will include 250-1500 mg of EPA, however any amount of EPA know to those of skill in the art that would counteract cytokine production and reduce muscle protein breakdown is contemplated in this disclosure.

The buffering agent included in an embodiment of the composition may comprise any buffering agent known to those of skill in the art that can reduce the likelihood of blood acidity. It is well known to those of skill in the art that administration of essential amino acids results in an increase in blood acidity and markers of bone resorption. The inclusion of the buffering agent in the disclosed composition generally enhances blood buffering capacity and minimizes the effects of the sulfur-containing amino acid, methionine, during formula intake. In addition, minimizing acidity will further augment the anabolic action of the EAAs. While any amount of any buffering agent known to those of skill in the art to reduce blood acidity is contemplated in this disclosure, in one embodiment of the disclosed composition the buffering agent element is comprised of 100-700 mg of sodium bicarbonate.

In application, the disclosed composition is intended for use in stressed patients and circumstances where renal insufficiency exists or may arise. Use is also contemplated in renal insufficient patients or others requiring HD. Because it is generally formulated to reduce skeletal muscle loss, maximize protein intake and address the potential problems of urea production and blood acidity, the disclosed composition is intended for use as longitudinal nutritional therapy for patients who develop renal insufficiency. For those in the latter stages of renal failure (i.e., CKD stage 5), compositions described herein are also intended for use prior to or with HD treatment.

Further use of the compositions are contemplated in any condition where renal insufficiency may develop and subsequently lead to a progressive muscle loss and inflammation, such as critical illness, sepsis, severe injury such as burns, advanced cancer, congestive heart failure, or chronic kidney disease itself.

The essential nutritional components of the composition can be provided as part of a pharmaceutical composition or nutritional (food) supplement for ease of consumption, or may be provided alone. When the composition is in the form of a food (or nutritional) supplement, the latter comprises, for example, a palatable base which acts as a vehicle for administering the composition to an individual and which can mask any unpleasant taste or texture of the composition. The food supplement may contain any one or several nutrients including drugs, vitamins, herbs, hormones, enzymes and/or other nutrients. The nutritional supplement may contain plural parts, where each of the plural parts is chronologically appropriate for its scheduled time of consumption.

In an embodiment, the food product may be a drink. Non-limiting examples of a suitable drink include fruit juice, a fruit drink, an artificially flavored drink, an artificially sweetened drink, a carbonated beverage, a sports drink, a liquid diary product, a shake, and so forth. The compositions may also be in liquid dosage forms for oral administration. Liquid dosage forms include aqueous and nonaqueous solutions, emulsions, suspensions and solutions and/or suspensions reconstituted from non-effervescent granules, containing suitable solvents, preservatives, emulsifying agents, suspending agents, diluents, sweeteners, coloring agents, and flavoring agents.

When the composition is in the form of a pharmaceutical composition, it can be administered in conventional form for oral administration. Solid dosage forms for oral administration may include capsules, tablets, caplets, pills, troches, lozenges, powders, and granules. A capsule typically comprises a core material comprising a composition of the invention and a shell wall that encapsulates the core material. The core material may be solid, liquid, or an emulsion. The shell wall material may comprise soft gelatin, hard gelatin, or a polymer. Suitable polymers include, but are not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, methacrylic acid, methyl acrylate, ammonio methylacrylate, ethyl acrylate, methyl methacrylate and/or ethyl methacrylate (e.g., those copolymers sold under the trade name “Eudragit”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified Iac). Some such polymers may also function as taste-masking agents.

Tablets, pills, and the like may be compressed, multiply compressed, multiply layered, and/or coated. The coating may be single or multiple. In one embodiment, the coating material may comprise a polysaccharide or a mixture of saccharides and glycoproteins extracted from a plant, fungus, or microbe. Non-limiting examples include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum. In another embodiment, the coating material may comprise a protein. Suitable proteins include, but are not limited to, gelatin, casein, collagen, whey proteins, soy proteins, rice protein, and corn proteins. In an alternate embodiment, the coating material may comprise a fat or oil, and in particular, a high temperature melting fat or oil. The fat or oil may be hydrogenated or partially hydrogenated, and preferably is derived from a plant. The fat or oil may comprise glycerides, free fatty acids, fatty acid esters, or a mixture thereof. In still another embodiment, the coating material may comprise an edible wax. Edible waxes may be derived from animals, insects, or plants. Non-limiting examples include beeswax, lanolin, bayberry wax, carnauba wax, and rice bran wax. Tablets and pills may additionally be prepared with enteric coatings.

Tablets and capsules for oral administration are usually presented in a unit dose, and contain conventional excipients such as binding agents, fillers, diluents, tabletting agents, lubricants, disintegrants, colourants, flavourings, and wetting agents. The tablets may be coated according to well-known methods in the art.

Suitable fillers for use include, mannitol and other similar agents. Suitable disintegrants include starch derivatives such as sodium starch glycollate. Suitable lubricants include, for example, magnesium stearate.

These solid oral compositions may be prepared by conventional methods of blending, filling, tabletting or the like. Repeated blending operations may be used to distribute the active agents throughout those compositions employing large quantities of fillers. Such operations are, of course, conventional in the art.

In certain cases it may be preferred to formulate the composition as an oral liquid preparation such as a syrup. The medicament can also be administered parenterally, e.g. by intramuscular or subcutaneous injection, using formulations in which the medicament is employed in a saline or other pharmaceutically acceptable, injectable composition.

Oral liquid preparations may be in the form of, for example, aqueous or oily suspensions, solutions, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethyl cellulose, aluminium stearate gel or hydrogenated edible fats, emulsifying agents, for example lecithin, sorbitan monooleate, or acacia; non-aqueous vehicles (which may include edible oils), for example, almond oil, fractionated coconut oil, oily esters such as esters of glycerine, propylene glycol, or ethyl alcohol; preservatives, for example methyl or propyl p-hydroxybenzoate or sorbic acid, and if desired conventional flavoring or coloring agents.

Oral formulations further include controlled release formulations, which may also be useful. The controlled release formulation may be designed to give an initial high dose of the active material and then a steady dose over an extended period of time, or a slow build up to the desired dose rate, or variations of these procedures. Controlled release formulations also include conventional sustained release formulations, for example tablets or granules having an enteric coating.

Nasal spray compositions are also a useful way of administering the pharmaceutical preparations to patients such as children for whom compliance may be difficult and may be used in an embodiment of the composition. Such formulations are generally aqueous and are packaged in a nasal spray applicator, which delivers a fine spray of the composition to the nasal passages.

Suppositories are also a traditionally good way of administering drugs to children and can be used in an embodiment of the composition. Typical bases for formulating suppositories include water-soluble diluents such as polyalkylene glycols and fats, e.g. cocoa oil and polyglycol ester or mixtures of such materials.

For parenteral administration, fluid unit dose forms are generally prepared containing the compound and a sterile vehicle. The compound, depending on the vehicle and the concentration, can be either suspended or dissolved. Parenteral solutions are normally prepared by dissolving the compound in a vehicle and filter sterilizing before filling into a suitable vial or ampoule and sealing. Advantageously, adjuvants such as a local anesthetic, preservatives and buffering agents are also dissolved in the vehicle.

Parenteral suspensions are prepared in substantially the same manner except that the compound is suspended in the vehicle instead of being dissolved and sterilized usually by exposure to ethylene oxide before suspending in the sterile vehicle.

Advantageously, a surfactant or wetting agent is included in the composition to facilitate uniform distribution of the compound of the invention.

As is common practice, the compositions will usually be accompanied by written or printed directions for use in the medical treatment concerned.

Properties of the disclosed composition are further illustrated by the following examples, which should not be construed as limiting.

Studies were undertaken to evaluate the effectiveness of EAAs on skeletal muscle protein anabolism. These studies also determined the effects of longitudinal administration on the accretion of lean body mass and muscle function. In addition, studies were performed in renal-insufficient patients, CKD patients in particular, to ascertain protein metabolism before and during HD. Also ascertained during the studies were the effects of correcting blood acidity on skeletal muscle protein metabolism.

### Example 1

To demonstrate the advantage of EAAs over traditional high quality proteins, muscle protein metabolism was examined in elderly subjects before and after the ingestion of whey protein or EAAs. Muscle protein kinetics were calculated before and for 3.5 hours following the bolus oral ingestion of 15 g EAAs (N=7) or 15 g whey protein (N=8) in elderly human subjects. Net phenylalanine uptake, an indication of net protein balance, over the post-supplement period was significantly greater for the EAA group compared to the whey group, as depicted graphically in the chart of FIG. 1 (P<0.05; 53±10 mg phe/leg EAA vs 21±5 mg phe/leg whey). While, both supplements stimulated muscle protein synthesis (p<0.05), the increase was greatest in the EAA group. The post-prandial rate of muscle protein synthesis was 0.088±0.011%.hr-1 for the EAA group, and 0.066±0.004%.hr-1 for the whey group (p<0.05). The greater increase in protein synthesis was due in part to the large increase in peripheral amino acid concentrations that result from free-form amino acid ingestion. The conversion of phenylalanine uptake to mg of protein results in an accrual of 4.0±0.4 g of protein/leg for the EAA blend, versus 2.2±0.3 g protein/leg for the whey protein, indicating that the 15 g EAA blend provided a much greater anabolic stimulus than the whey protein supplement in the elderly test group.

Not only was the EAA blend more effective, but the efficiency of protein utilization (net protein synthesis/protein [i.e., AA] ingestion) was approximately 1.1 for the EAA mixture as opposed to approximately 0.2 for whey protein. The value of 0.2 for whey protein is consistent with recorded observations in the art (See, e.g., Hegsted D M. Assessment of nitrogen requirements. Am J Clin Nutr 1978; 31(9):1669-77) in that about 20% of nitrogen intake above requirement for balance is retained in the body. The four-fold higher ratio for the EAAs reflects an efficient reutilization of non-essential amino acids that otherwise would have been wasted/excreted. This ratio also reflects an optimal formulation of an EAA mixture to match the requirement for muscle protein synthesis. In addition, this formula has the added benefit of stimulating muscle protein synthesis with less than half of the total amino acids that would be derived from an intact protein. For the latter stage CKD patient, these results indicate that substituting intact protein with EAA will result in a greater benefit per unit intake.

### Example 2

To demonstrate the effects of EAA on blood urea production, subjects were given two doses of 6 g EAAs one hour apart after completion of a bout of resistance exercise. Acute changes in the rate of urea production were measured using a tracer technique known to those of skill in the art. Despite ingestion of a total of 12 g of EAAs, urea production did not increase and, in fact, trended downward, as graphically depicted in the chart of FIG. 2A. Using the same tracer methodology, it was demonstrated that an infusion of alanine or glutamine stimulated urea significantly over the same time interval. The reason for the lack of an increase in urea production is believed to be the reutilization of non-essential amino acids for muscle protein synthesis rather than transport to the liver for degradation and incorporation of the nitrogen into urea. This is reflected by the steady drop in the concentration of alanine in the blood, as depicted in the chart of FIG. 2B. Thus, the EAA blend greatly stimulates net muscle protein synthesis without increasing blood urea concentration. This aspect of the disclosed composition is of potentially great advantage to CKD patients, as the provision of intact protein alone increases blood urea while having minimal effect on muscle protein net balance.

### Example 3

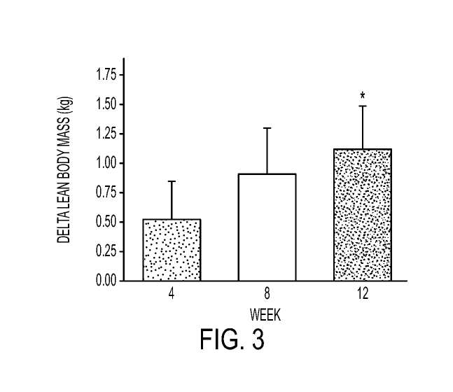

In order to demonstrate the efficacy of prolonged EAA supplementation on lean body mass (LBM) and functional outcomes, 12 elderly (67.0±5.6 [SD] years) subjects with impaired glucose tolerance who were given 11 g of EAA capsules BID for 12 weeks were studied. The subjects did not engage in a regular exercise program and dietary records indicated no change in dietary habits or intake. Following this structure, it was discovered that LBM was measured by duel energy x-ray absorptiometry (DEXA) at baseline and at weeks 4, 8, and 12. In addition, maximal leg strength and muscle function were tested at baseline and weeks 8 and 12. LBM increased steadily at each 4 week time point, reaching significance at 12 weeks, as depicted in the chart of FIG. 3. The increase of 1 kg of LBM, on average, is of potential benefit in terms of metabolic reserve in stressed patient populations.

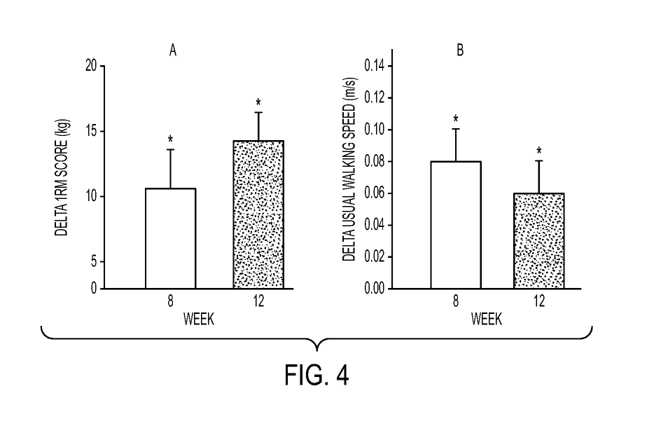

The increase in LBM alone is important to the CKD population, as it a strong predictor of morbidity and mortality. However, the translation of LBM to functional outcomes holds even greater promise. In the studied elderly impaired glucose tolerance subjects, a substantial increase in leg strength was noted, as depicted in the chart of FIG. 4A, as assessed by 1 Repetition Maximum (1RM; the maximal weight lifted one time), and function as depicted in the chart of FIG. 4B, as assessed by walking speed. In addition, the studied subjects significantly increased their scores on the 5-step and floor transfer tests. Thus, these subjects gained LBM, strength, and function without altering their dietary intake or exercise regimen. Thus, it was concluded that EAA stimulation of protein turnover results in not only a net increase in LBM, but the formation of more functional proteins which manifest in greater muscular strength and function.

This study is particularly applicable to latter stage CKD patients, as they tend to be older, less active, and insulin resistant. Insulin resistance in CKD patients is related to a defect in insulin signaling and leads to accelerated muscle degradation. This study demonstrates that prolonged administration of EAA in insulin-resistant subjects effectively increases LBM and, more importantly, translates to improved functional outcomes.

### Example 4

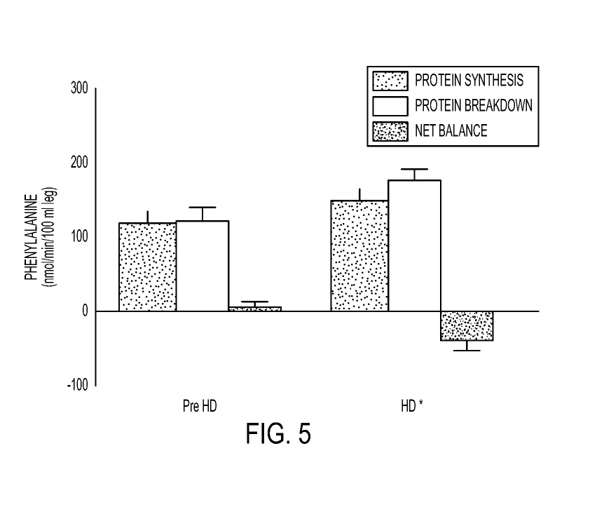

As mentioned in the disclosure, the greatest alteration in protein metabolism that leads to the loss of skeletal muscle in latter stage CKD patients is their constant exposure to HD. Six CKD Stage 5 patients before and after HD were studied. The study demonstrated that if patients consumed the recommended diet (35 kcal/kg, 1.2 g protein/kg/d) and a buffer was given to adjust blood bicarbonate to ≧22 meq/L, protein balance was achieved during a brief fast, as depicted in the chart of FIG. 5; Pre HD. However, after 3 hours of HD, the skeletal muscle became very catabolic. There was a coordinated increase in both synthesis and breakdown; however, breakdown increased to a greater degree such that the net protein balance was substantially catabolic, as depicted in the chart of FIG. 5; HD. The net release of amino acids from skeletal muscle during HD is required to support the increase in liver protein synthesis. The central provision of amino acids by oral ingestion of the disclosed composition before HD will minimize the requirement for peripheral (skeletal muscle) amino acid release. Greater splanchnic extraction will be advantageous in that adequate central precursors will be readily available and alleviate the requirement for amino acids from the periphery.

Data known to those of skill in the art shows that the fractional synthetic rates of albumin, fibrinogen, and muscle protein increases during HD by approximately 39%, 54%, and 53%, respectively (See, e.g., Raj D S, Dominic E A, Wolfe R, et al. Coordinated increase in albumin, fibrinogen, and muscle protein synthesis during hemodialysis: role of cytokines. Am J Physiol Endocrinol Metab 2004; 286(4):E658-64). Provision of 15 g of EAAs increases muscle protein synthesis by approximately 70%, whereas amino acid ingestion increases albumin synthesis by 50%. Thus, the compositions appear to support skeletal muscle protein synthesis and offset the negative balance in skeletal muscle, while simultaneously supporting the liver protein synthetic requirements.

Taken together, the compositions discussed herein addresses several important problems associated with renal insufficiency. First, they provide composition and administration methods for preserving skeletal muscle mass in stressed patients; the disclosed composition can be utilized to slow, reduce, or prevent the loss of lean body mass in patients who may develop renal insufficiency. In addition, the disclosed compositions appear to replace a substantial proportion of high quality protein intake, thereby resulting in a greater anabolic effect on muscle protein per gram of protein intake.

Second, the disclosed compositions address other key metabolic disorders in stressed patients, namely, increased blood acidity and urea production. Amino acid (protein) metabolism during renal insufficiency is problematic since the metabolites can not be excreted via urinary output. For example, the metabolism of sulfur-containing amino acids, such as methionine, increases blood acidity (lowers pH) via the formation of sulfuric acid. In addition, amino acid oxidation requires the formation of urea prior to excretion of the nitrogen component. However, the inability of renal-insufficient patients to excrete urea leads to additional problems associated with uremia and blood acidity.

Third, renal insufficiency entails a persistent state of inflammation, represented in part by a chronic elevation in cytokines. The inclusion of EPA in an embodiment of the composition should reduce the elevation in circulating cytokines. In addition, EPA also generally reduces muscle protein breakdown.

Finally, the combination of EAA and EPA will benefit renal-insufficient patients during HD by providing adequate amino acid precursors to sustain liver protein synthesis without the reliance on amino acids derived from skeletal muscle. Therefore, muscle protein breakdown will be diminished due to a decreased central requirement for muscle-derived amino acids, and due to the effect of EPA on muscle protein breakdown. Thus, a synergism is expected between the amino acid component and the EPA component. While the EAAs target the increase in muscle protein synthesis, EPA works to reduce muscle protein breakdown. Since net protein balance equals protein synthesis minus protein breakdown, the expected increase in protein synthesis and the concomitant decrease in protein breakdown will result in a greater positive net balance in these patients, thus maintaining lean body mass, functional capability, and better quality of life.

While the invention has been disclosed in connection with certain preferred embodiments, this should not be taken as a limitation to all of the provided details. Modifications and variations of the described embodiments may be made without departing from the spirit and scope of the invention, and other embodiments should be understood to be encompassed in the present disclosure as would be understood by those of ordinary skill in the art.

1. A composition of matter comprising:
An EAA blend, said EAA blend including: valine, threonine, isoleucine, leucine, lysine, phenylalanine, and methionine, and not including glutamine or alanine;
eicosapentaenoic acid (EPA);
a macronutrient blend, said macronutrient blend comprising macronutrients selected from the group consisting of: protein, carbohydrate, and fat; and
a buffering agent.

2. The composition of claim 1 wherein said EAA blend further comprises arginine and histidine.

3. The composition of claim 2 wherein:
histidine comprises 1-6% of said EAA blend;
isoleucine comprises 6-15% of said EAA blend;
leucine comprises 15-40% of said EAA blend;
lysine comprises 10-25% of said EAA blend;
methionine comprises 1-5% of said EAA blend;
phenylalanine comprises 5-15% of said EAA blend;
threonine comprises 5-15% of said EAA blend;
valine comprises 5-20% of said EAA blend; and
arginine comprises 5-15% of said EAA blend.

4. The composition of claim 1 further comprising citrulline.

5. The composition of claim 1 wherein:
carbohydrate comprises 30-60% of said macronutrient blend;
fat comprises 10-25% of said macronutrient blend; and
protein comprises 25-75% of said macronutrient blend.

6. The composition of claim 1 wherein said buffering agent comprises sodium bicarbonate.

7. The composition of claim 1 comprising:
about 15 grams of said EAA blend;
about 1 to about 15 grams of said macronutrient blend;
about 250 to about 1500 milligrams of EPA; and
about 100 to about 700 milligrams of buffering agent.

8. The composition of claim 1 wherein said macronutrient blend comprises protein peptides.

9. The composition of claim 1 wherein said macronutrient blend comprises whey protein.

10. The composition of claim 1 wherein said composition is part of a food product.

11. The composition of claim 9 wherein said food product comprises a drink.

12. The composition of claim 1 wherein said composition is part of a pharmaceutical preparation.

13. A composition of matter consisting essentially of:
valine; threonine; isoleucine; leucine; lysine; phenylalanine; methionine; histidine;
and arginine;

14. The composition of claim 13 wherein:
histidine comprises 1-6% of said composition;
isoleucine comprises 6-15% of said composition;
leucine comprises 15-40% of said composition;
lysine comprises 10-25% of said composition;
methionine comprises 1-5% of said composition;
phenylalanine comprises 5-15% of said composition;
threonine comprises 5-15% of said composition;
valine comprises 5-20% of said composition; and
arginine comprises 5-15% of said composition.

15. A composition of matter consisting essentially of:
an EAA blend consisting of: valine, threonine, isoleucine, leucine, lysine, phenylalanine, and methionine; histidine; and arginine;
eicosapentaenoic acid (EPA);
a macronutrient blend consisting of protein; carbohydrate; and fat; and
a buffering agent.

16. The composition of claim 15 wherein:
histidine comprises 1-6% of said EAA blend;
isoleucine comprises 6-15% of said EAA blend;
leucine comprises 15-40% of said EAA blend;
lysine comprises 10-25% of said EAA blend;
methionine comprises 1-5% of said EAA blend;
phenylalanine comprises 5-15% of said EAA blend;
threonine comprises 5-15% of said EAA blend;
valine comprises 5-20% of said EAA blend; and
arginine comprises 5-15% of said EAA blend.

17. The composition of claim 15 wherein:
carbohydrate comprises 30-60% of said macronutrient blend;
fat comprises 10-25% of said macronutrient blend; and
protein comprises 25-75% of said macronutrient blend.

18. The composition of claim 15 wherein said buffering agent comprises sodium bicarbonate.

19. A method of inhibiting skeletal muscle degradation during renal failure, the method comprising:
administering to said individual a therapeutically effective amount of a composition comprising:
an EAA blend comprising: valine, threonine, isoleucine, leucine, lysine, phenylalanine, and methionine; histidine; and arginine;
eicosapentaenoic acid (EPA);
a macronutrient blend consisting of protein; carbohydrate; and fat; and
a buffering agent; and
administering hemodialysis to said individual.

20. A composition of matter consisting essentially of:
an EAA blend consisting of valine, threonine, isoleucine, leucine, lysine, phenylalanine, and methionine; histidine; and arginine; and
eicosapentaenoic acid (EPA).
